(19) United States
(12) Patent Application Publication
(10) Pub. No.: US 2006/0194285 A1
(43) Pub. Date: Aug. 31, 2006

(54) Biological production of clavulanic acid and related compounds

(76) Inventors: Wei Lian, Worcester, MA (US); Wei-Shou Hu, Falcon Heights, MN (US); David H. Sherman, Ann Arbor, MI (US)

Correspondence Address: MUETING, RAASCH & GEBHARDT, P.A., P.O. BOX 581415, MINNEAPOLIS, MN, 55458, US

(21) Appl. No.: 11/348,838

(22) Filed: Feb. 7, 2006

Related U.S. Application Data
(60) Provisional application No. 60/650,783, filed on Feb. 8, 2005.

(51) Int. Cl. C12N 9/10; C07H 21/04; C12P 21/06; C12N 15/74; C12N 1/21

(52) U.S. Cl. 435/69.1; 435/252.35; 435/193; 435/471; 536/23.2

(57) ABSTRACT

Disruption of the two-component regulatory system encoded by the cbsA locus is associated with improved production of cephamycin C and/or clavulanic acid in Streptomyces clavuligerus.

### CROSS REFERENCE TO RELATED APPLICATION

This application claims the benefit of U.S. Provisional Application Ser. No. 60/650,783, filed Feb. 8, 2005, which is incorporated herein by reference in its entirety.

### STATEMENT OF GOVERNMENT RIGHTS

The present invention was made with support by the National Institutes of Health, Grant No. GM55850. The government may have certain rights in the invention.

### BACKGROUND

Streptomyces species are soil bacteria characterized by their complex morphological development and propensity to produce a diverse range of secondary metabolites. The production of those biologically active natural products by Streptomycetes is often responsive to various environmental signals.

Control of antibiotic production occurs through a complex network of cellular signals, but invariably involves both pathway-specific and global regulators of gene expression. Pathway-specific regulators exist widely in many antibiotic biosynthetic gene clusters in different species. They have been shown to directly and specifically control expression of genes in a particular secondary metabolic pathway, and the pathway-specific regulator gene is often physically located within the system it controls. Global regulators, on the other hand, generally work on a higher level of the regulatory hierarchy, and can affect biosynthesis of several genetically and structurally distinct antibiotics. This type of regulator may or may not be located within the antibiotic gene cluster that it controls.

The absA locus from Streptomyces coelicolor encodes a two-component global regulatory system. It affects antibiotic production of at least three distinct biosynthetic pathways, including actinorhodin, undecylprodigiosin and calcium-dependent antibiotic (CDA). The locus was originally thought to be a positive regulator because a reduced antibiotic phenotype was observed in a strain bearing a UV-induced mutation within the absA locus. However, when the genes of this two-component system were disrupted, mutants showed greatly enhanced antibiotic production. Thus, the two-component system encoded by absA was considered to exert negative control on antibiotic biosynthesis in S. coelicolor. Genotypic analysis of several mutants with variant point mutations in absA revealed an unpredictable relationship between phenotype and genotype which indicated the mechanistic complexity of the two-component system. Completion of the genome sequencing project for S. coelicolor has shown that absA is located within the CDA biosynthetic gene cluster, and subsequent studies revealed that absA positively controls some promoters and negatively regulates others within the CDA system. Interruption of the absA locus in the chromosomal DNA of S. coelicolor has been shown to cause hyperproduction of the antibiotics actinorhodin and undecylprodigiosin (U.S. Pat. No. 5,876,987, issued Mar. 2, 1999, Champness et al.).

Additional two-component systems that affect antibiotic production have been identified and studied. CutR/S was first identified in Streptomyces lividans, a closely related species to S. coelicolor. The system negatively regulates secondary metabolism in both S. lividans and S. coelicolor. AfsQ1/2 plays a subtle role in regulation of antibiotic biosynthesis in wild-type S. coelicolor but can suppress the phenotype of absA mutants when transformed on a high copy number plasmid. The phoR/phoP genes exist both in S. coelicolor and S. lividans. The knock-out S. lividans mutants lost the ability to express extracellular alkaline phosphatase but were able to increase both actinorhodin and undecylprodigiosin production significantly. However, two-component systems are abundant in Streptomyces species and not all of them are either directly or indirectly involved in regulation of antibiotic biosynthesis. The complete S. coelicolor genome sequence revealed 85 sensor kinase genes and 79 response regulator genes, including 53 sensor-regulator pairs. See, e.g., Hutchings et al., Microbiol., 2004, September: 150 (Pt 9):2795-2806. ChiS/ChiR was shown to be involved in regulating chitinase production; CseB/CseC is related to cell wall damage; VanR/VanS controls cellular resistance to vancomycin.

S. clavuligerus produces two particularly important natural products—cephamycin C and clavulanic acid. Cephamycin C belongs to the β-lactam antibiotic family that includes penicillin G and cephalosporin C. β-lactam antibiotics act by inhibiting formation of the bacterial cell wall, thereby killing bacteria. However, some bacteria have acquired genes encoding enzymes called β-lactamase, which can inactivate β-lactam antibiotics, causing a resistance phenotype. Thus the efficacy of this type of antibiotics is drastically reduced by the action of β-lactamase enzymes. Clavulanic acid, sharing a similar chemical structure as the β-lactam antibiotics, works as a β-lactamase inhibitor by binding to the enzyme irreversibly. The combination of clavulanic acid and β-lactam antibiotics, therefore, can overcome the resistance developed by bacteria. AUGMENTIN® (GlaxoSmithKline) and its generic equivalent is the most prescribed anti-infective agent containing a combination of antibiotic and β-lactamase inhibitor including a mixture of amoxicillin and clavulanic acid. It has a market value of more than one billion dollars yearly.

The gene clusters that specify biosynthesis of clavulanic acid and cephamycin C have been shown to reside immediately adjacent to one another. In each cluster there is a pathway-specific regulator including CcaR for cephamycin C biosynthesis and ClaR for clavulanic acid biosynthesis. Both gene products are positive regulators for the corresponding clusters. However, while ClaR specifically regulates clavulanic acid production, CcaR regulates both cephamycin C and clavulanic acid production. Experimental evidence suggests that CcaR regulates clavulanic acid levels by affecting directly claR expression.

S. clavuligerus is thus a well-characterized and important source of bioactive metabolites, including various antibiotics as well as the important β-lactamase inhibitor clavulanic acid. Methods for increasing the production of bioactive metabolites, particularly clavulanic acid, in S. clavuligerus would constitute an important advance in the fight against infectious diseases.

### SUMMARY OF THE INVENTION

The invention relates to a newly discovered two-component global regulatory system in Streptomyces clavuligerus which is encoded by the cbsA locus. Like the absA locus of Streptomyces coelicolor, this two-component system includes a response regulator (cbsA2) and a sensor kinase (cbsA1). It is involved in regulation of the biosynthesis of the bioactive metabolites cephamycin C and clavulanic acid. Clavulanic acid, a β-lactamase inhibitor, is a key component of the AUGMENTIN (and its generic equivalent co-amoxiclav) anti-infective agent, one of the most effective and widely used antibacterial drugs in clinical use. Disruption of this two-component regulatory system was found to enhance the production of cephamycin C and clavulanic acid by S. clavuligerus.

In one aspect, the invention provides a genetically modified Streptomyces, preferably a S. clavuligerus, that exhibits enhanced production of a bioactive metabolite compared to the level of production of the bioactive metabolite in a comparable wild-type Streptomyces, preferably S. clavuligerus. In one embodiment, the bioactive metabolite is a member of the cephalosporin family. Preferably, the bioactive metabolite is a cephamycin, such as cephamycin C, or a clavulanic acid.

In one embodiment, the genetically modified Streptomyces includes a structural alteration in the cbsA locus that reduces or eliminates the production or activity of a gene product, e.g., an RNA or a polypeptide, of cbsA1 or cbsA2. In another embodiment, the genetically modified Streptomyces includes a structural alteration in a different part of its genome that directly or indirectly reduces or eliminates the production or activity of a gene product of cbsA1 or cbsA2, such as a structural alteration in a regulatory region that is not positioned within the cbsA locus. The polypeptide gene product is preferably a sensor kinase polypeptide encoded by cbsA1 or a response regulator polypeptide encoded by cbsA2.

The structural alteration can include, for example, a deletion, insertion or substitution in the cbsA locus, or other region that affects the production of one or more gene products of the cbsA locus, and is preferably present in either or both the genes cbsA1 or the cbsA2. In a particularly preferred embodiment, the structural alteration includes a deletion, insertion or substitution in the coding region of either or both of cbsA1 or the cbsA2. For example, the invention provides a genetically modified S. clavuligerus that produces a bioactive metabolite and has a cbsA locus which has been modified by deletion of a segment of the DNA of the cbsA locus or insertion of a foreign DNA in the cbsA locus or both. In a preferred embodiment, the genetically modified S. clavuligerus includes a cbsA locus that has been modified by disruption of a segment of the DNA of the cbsA locus so that there is hyperproduction of the bioactive metabolite.

In another aspect, the invention provides a biological method for making a bioactive metabolite. In one embodiment, the bioactive metabolite is from cephalosporin family. Preferably, the bioactive metabolite is cephamycin or a clavulanic acid. In a preferred embodiment, the method increases or enhances the production of a naturally occurring bioactive metabolite in Streptomyces, preferably S. clavuligerus. An increase or enhancement of the production of the bioactive metabolite is achieved by reducing or eliminating the production or activity of an RNA or polypeptide gene product encoded by cbsA1 or cbsA2.

In one embodiment, the method for making the bioactive metabolite includes culturing the genetically modified Streptomyces, preferably S. clavuligerus, of the invention under conditions and for a time sufficient to produce the bioactive metabolite, and isolating the bioactive metabolite.

In another embodiment, the method for making the bioactive metabolite includes introducing an exogenous agent into a Streptomyces, preferably a S. clavuligerus, to yield a modified Streptomyces, preferably S. clavuligerus, wherein the exogenous agent reduces or eliminates the production or activity of a gene product of cbsA1 or cbsA2. The modified Streptomyces is cultured under conditions and for a time sufficient to produce the bioactive metabolite, and the bioactive metabolite is isolated. The invention is not limited by the nature of the exogenous agent supplied, which can be, for example, a DNA, RNA or small molecule inhibitor. The exogenous agent can be introduced before or during the culturing of the modified Streptomyces.

In another aspect, the invention provides method for making a modified Streptomyces of the invention, preferably a modified S. clavuligerus, that includes modifying a Streptomyces cell to reduce or eliminate the production or activity of a gene product of cbsA1 or cbsA2. In one embodiment, the method includes structurally altering the cbsA locus to yield a genetically modified Streptomyces, preferably a genetically modified S. clavuligerus, for example by effecting a deletion, insertion or substitution in the cbsA locus. The structural alteration is preferably made within a coding region of the cbsA locus, although it can be made at other locations in the cbsA locus as well. A particularly preferred method for structurally altering the cbsA locus includes inserting a foreign DNA into the cbsA locus. Alternatively, the structural alteration can be made in a different part of the Streptomyces genome, so that it directly or indirectly reduces or eliminates the production or activity of a gene product of cbsA1 or cbsA2. For example, a structural alteration can be made in a regulatory region that is not positioned within the cbsA locus.

In another embodiment, the method for making a modified Streptomyces of the invention includes introducing an exogenous agent into a Streptomyces cell, preferably a S. clavuligerus cell, wherein the exogenous agent reduces or eliminates the production or activity of a gene product of cbsA1 gene or the cbsA2 gene.

In another aspect, the invention provides an isolated polypeptide comprising an amino acid sequence having at least 80% identity, more preferably at least 90% identity, to SEQ ID NO: 3 or SEQ ID NO:5. An isolated polynucleotide that encodes a polypeptide of the invention is also provided. In a particularly preferred embodiment, the invention provides a polynucleotide that encodes a polypeptide having SEQ ID NO: 3 or SEQ ID NO:5 or a biologically active analog or subunit thereof. The polynucleotide of the invention preferably includes a nucleotide sequence having at least 90% identity to SEQ ID NO: 2 or SEQ ID NO:4. Complements of the polynucleotides described herein are also provided.

Unless otherwise specified, “a,” “an,” “the,” and “at least one” are used interchangeably and mean one or more than one.

### BRIEF DESCRIPTION OF THE DRAWINGS

FIG. 1A shows the organization of the cbsA2/cbsA1 open reading frames (ORFs) on the chromosome; below the ORFs are the corresponding conserved protein motifs that are characteristic of two-component systems.

FIG. 1B shows an alignment of amino acid sequences encoded by absA1 from S. coelicolor and cbsA1 from S. clavuligerus.

FIG. 1C shows an alignment of amino acid sequences encoded by absA2 from S. coelicolor and cbsA2 from S. clavuligerus.

FIG. 2 shows construction of S. clavuligerus DHS3507 carrying ΔcbsA2::kan and DHS3508 carrying ΔcbsA1::kan on the chromosome. Disruption of cbsA2 and cbsA1 was accomplished by replacing part of each gene with a kanamycin resistance gene.

FIG. 3 shows growth kinetics, pH, cephamycin titer and clavulanic acid titer in modified TSB liquid medium of S. clavuligerus wild type versus DHS3507 in (a) and wild type versus DHS3508 in (b).

FIG. 4 shows Northern blotting of total RNA from S. clavuligerus with ccaR and claR probes. The 16S rRNA gel data indicates the same total amount of RNA was loaded. Lane 1 and lane 2 are from wild type and DHS3507 at 35 hours from the culture shown in FIG. 3(a); Lane 3 and 4 are from wild type and DHS3508 at 50 hours from culture shown in FIG. 3(b).

FIG. 5 shows the nucleotide sequence for the cbsA locus, including both cbsA1 and cbsA2 (SEQ ID NO:1). The open reading frame for cbsA2 runs from nucleotide 524 through nucleotide 1219, and the open reading frame for cbsA1 runs from nucleotide 1308 through nucleotide 2945.

FIG. 6 shows the nucleotide sequence open reading frame for cbsA1 (SEQ ID NO:2).

FIG. 7 shows the amino acid sequence encoded by the open reading frame for cbsA1 (SEQ ID NO:3).

FIG. 8 shows the nucleotide sequence open reading frame for cbsA2 (SEQ ID NO:4).

FIG. 9 shows the amino acid sequence encoded by the open reading frame for cbsA2 (SEQ ID NO:5).

### DETAILED DESCRIPTION OF ILLUSTRATIVE EMBODIMENTS

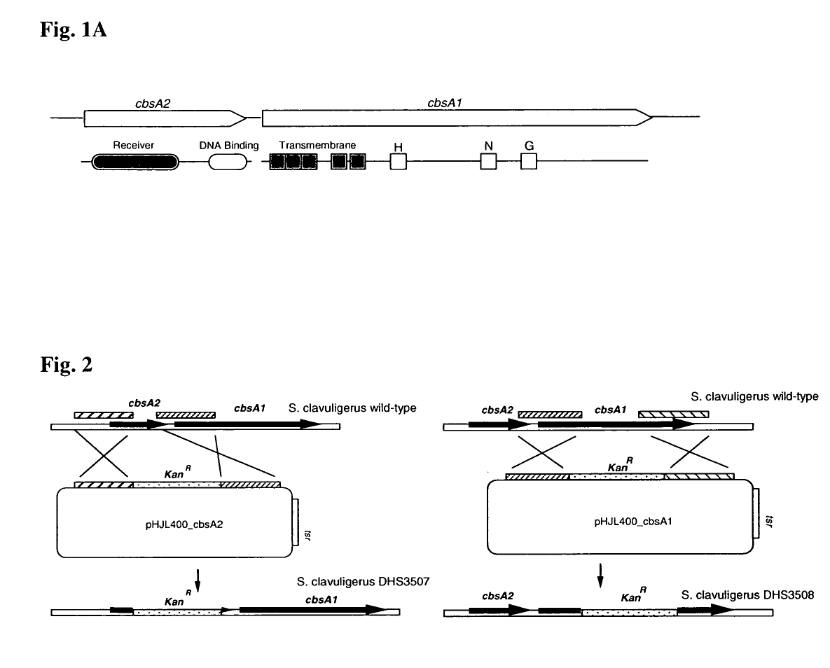

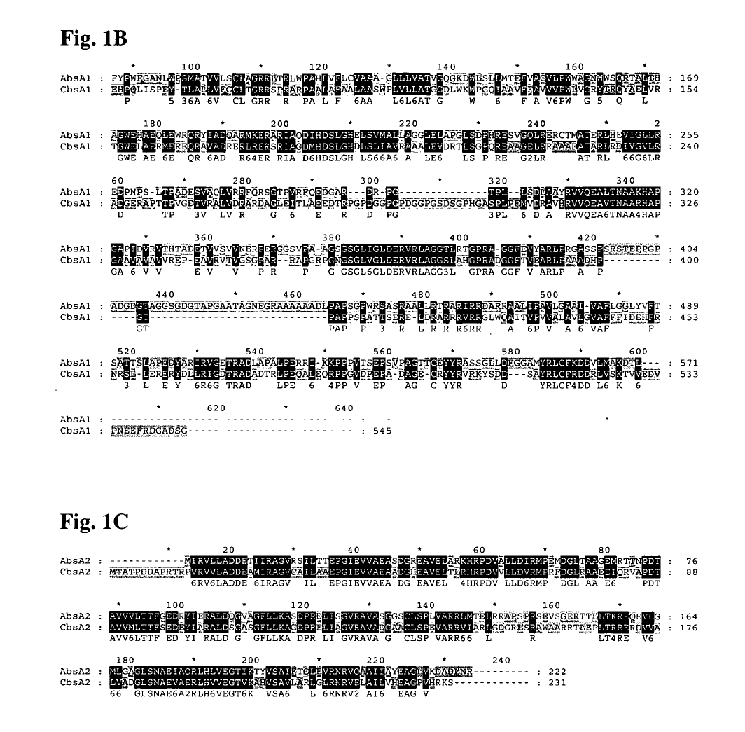

The newly discovered two-component global regulatory system in Streptomyces clavuligerus, encoded by the cbsA locus, includes a response regulator (cbsA2) and a sensor kinase (cbsA1) (FIG. 1A). This global system was found to be involved in regulation of the biosynthesis of the bioactive metabolites cephamycin C and clavulanic acid. Although previous studies have shown that antibiotic production in S. clavuligerus can be increased by manipulating pathway-specific regulatory genes (e.g. cephamycin C; Alexander et al., J. Bacteriol. 1998, 180, 4068-4079; Alexander et al., J. Bacteriol. 2000, 182, 348-356), this is the first demonstration in S. clavuligerus of a two-component regulatory system that can be manipulated to increase the production of clavulanic acid.

It was found that disruption of the two-component regulatory system encoded by the cbsA locus resulted in improved production of the antibiotic cephamycin C and/or the β-lactamase inhibitor clavulanic acid by S. clavuligerus. This was surprising, as our studies of Streptomyces have revealed that only a small portion of about 20 two-component systems that we disrupted had any effect on antibiotic production, and such effect could not be predicted in advance. In the case of the cbsA locus of S. clavuligerus, we further observed that improvement in production (hyperproduction) resulted from the disruption of either or both of the cbsA2 and cbsA1 genes. In addition to cephamycin C and/or clavulanic acid, improved production is expected for any member of the cephalosporin family of bioactive metabolites which is produced in the cephamycin metabolic pathway. For example, gene expression is upregulated, which will increase production of late stage metabolites including cephalosporin, a precursor to cephamycin C in S. clavuligerus. Other antibiotics produced by S. clavuligerus include anti-podal clavams, holomycin and tunicamycin (de la Fuente et al., J. Bacteriol., December 2002, 184:6559-6565).

This technology thus identifies new mutation targets, in the form of the newly discovered regulatory genes cbsA2 and cbsA1 and their gene products (RNAs and polypeptides), within Streptomyces spp. such as S. clavuligerus to enhance clavulanic acid production. In that regard it is important to note that, although the cbsA locus of the invention was discovered in S. clavuligerus, the invention is not limited to S. clavuligerus and methods of making and using S. clavuligerus, but should be understood to relate also to other Streptomyces species that express sensor kinases and response regulator polypeptides as described herein, as well as methods of making and using such other Streptomyces spp.

It should be noted that the cbsA locus discovered in S. clavuligerus differs from the absA locus in S. coelicolor in a number of important respects. For example, the relative position of the regulatory genes cbsA2 and cbsA1 is reversed in comparison to the positions of absA2 and absA1 in the S. coelicolor absA locus. The cbsA locus is located in a different part of the genome compared to the absA locus. Additionally, the sequence identity between the amino acid sequences encoded by cbsA2 and absA2 is only about 72% (FIG. 1C) and the sequence identity between the amino acid sequences encoded by cbsA1 and absA1 is only about 52% (FIG. 1B).

Also, other genes located outside the antibiotic biosynthetic pathway have been recently observed to affect production of clavulanic acid, including bldG (Bignell et al., 2005, Antimicrob. Agents Chemother. 49:1529-1541) and relA/spoT (Jin et al., 2004, Microbiol. 150:1485-1493). Disruption of any of these genes in combination with disruption of either or both of the cbsA2 and cbsA1 genes is expected to further enhance the production of cephamycin C and/or clavulanic acid.

In one aspect, the invention provides novel polypeptides. If naturally occurring, the polypeptide is preferably isolated from its natural environment and purified. In one embodiment, the invention provides a sensor kinase polypeptide (e.g., CbsA1 from S. clavuligerus; SEQ ID NO:3). In another embodiment, the invention provides a response regulator polypeptide (e.g., CbsA2 from S. clavuligerus; SEQ ID NO:5). Biologically active analogs and subunits of these polypeptides are included in the invention as well. Polynucleotides encoding these polypeptides, as well as biologically active analogs and subunits thereof, are also encompassed. Exemplary polynucleotides include a polynucleotide having a nucleotide sequence represented by SEQ ID NO: 1 (cbsA locus), SEQ ID NO:2 (cbsA1 open reading frame) and SEQ ID NO:4 (cbsA2 open reading frame).

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor do are they intended to imply or distinguish whether the polypeptide is produced using recombinant techniques, chemical or enzymatic synthesis, or is naturally occurring.

Likewise, the term “polynucleotide” refers broadly to a polymer of two or more nucleotides covalently linked in a 5′ to 3′ orientation. The terms nucleic acid, nucleic acid molecule, and oligonucleotide and polynucleotide included within the definition of polynucleotide and these terms are used interchangeably. It should be understood that these terms do not connote a specific length of a polymer of nucleotides, nor do are they intended to imply or distinguish whether the polynucleotide is produced using recombinant techniques, chemical or enzymatic synthesis, or is naturally occurring.

Polynucleotides can be single-stranded or double-stranded, and the sequence of the second, complementary strand is dictated by the sequence of the first strand. The term “polynucleotide” is therefore to be broadly interpreted as encompassing a single stranded nucleic acid polymer, its complement, and the duplex formed thereby. “Complementarity” of polynucleotides refers to the ability of two single-stranded polynucleotides to base pair with each other, in which an adenine on one polynucleotide will base pair with a thymidine (or uracil, in the case of RNA) on the other, and a cytidine on one polynucleotide will base pair with a guanine on the other. Two polynucleotides are complementary to each other when a nucleotide sequence in one polynucleotide can base pair with a nucleotide sequence in a second polynucleotide. For instance, 5′-ATGC and 5′-GCAT are fully complementary, as are 5′-GCTA and 5′-TAGC.

In the case of a polypeptide or polynucleotide that is naturally occurring, it is preferred that such polypeptide or polynucleotide be isolated and purified. An “isolated” polypeptide or polynucleotide is one that is separate and discrete from its natural environment. A “purified” polypeptide or polynucleotide is one that is at least 60% free, preferably 75% free, and most preferably 90% free from other components with which they are naturally associated. Polypeptides and nucleotides that are produced outside the organism in which they naturally occur, e.g., through chemical or recombinant means, are considered to be isolated and purified by definition, since they were never present in a natural environment.

A “biologically active” analog, subunit or derivative of a CbsA1 polypeptide is a polypeptide that exhibits sensor kinase activity. A “biologically active” analog, subunit or derivative of a CbsA2 polypeptide is a polypeptide that exhibits response regulator activity. Examples of methods for measuring activities can be found in Sheeler et al., J. Bacteriol. 2005, 187, 687-696.

A biologically active “subunit” of a polypeptide of the invention includes a CbsA1 polypeptide or a CbsA2 polypeptide that has been truncated at either the N-terminus, or the C-terminus, or both, by one or more amino acids, as long as the truncated polypeptide retains bioactivity and contains at least 7 amino acids, more preferably at least 10 amino acids, most preferably at least 12 amino acids.

A biologically active “analog” of a polypeptide of the invention includes a CbsA1 or a CbsA2 polypeptide that has been modified by the addition, substitution, or deletion of one or more contiguous or noncontiguous amino acids, or that has been chemically or enzymatically modified, e.g., by attachment of a reporter group, by an N-terminal, C-terminal or other functional group modification or derivatization, or by cyclization, as long as the analog retains biological activity. An analog can thus include additional amino acids at one or both of the termini of a polypeptide.

Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For the purposes of this invention, conservative amino acid substitutions are defined to result from exchange of amino acids residues from within one of the following classes of residues: Class I: Ala, Gly, Ser, Thr, and Pro (representing small aliphatic side chains and hydroxyl group side chains); Class II: Cys, Ser, Thr and Tyr (representing side chains including an —OH or —SH group); Class III: Glu, Asp, Asn and Gln (carboxyl group containing side chains): Class IV: His, Arg and Lys (representing basic side chains); Class V: Ile, Val, Leu, Phe and Met (representing hydrophobic side chains); and Class VI: Phe, Trp, Tyr and His (representing aromatic side chains). The classes also include related amino acids such as 3Hyp and 4Hyp in Class I; homocysteine in Class II; 2-aminoadipic acid, 2-aminopimelic acid, γ-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoquinoline-3-carboxylic acid, and halogenated tyrosines in Class VI.

Preferred biologically active analogs of CbsA1 or CbsA2 or any of their constituent peptides include those analogs that are at least 80% identical, more preferably 85% identical, more preferably at least 90% identical, even more preferably at least 95% identical, and most preferably at least 96%, 97%, 98% or 99% identical to CbsA1 or CbsA2 or their constituent peptides. Such analogs contain one or more amino acid deletions, insertions, and/or substitutions relative to CbsA1 or CbsA2, and may further include chemical and/or enzymatic modifications and/or derivatizations as described above.

Preferred polynucleotides of the invention include polynucleotides encoding polypeptide sequences SEQ ID NO:3 or 5, and the complement of such polynucleotide, as well as polynucleotides having a nucleotide sequence that is “substantially complementary” to (a) a nucleotide sequence that encodes polypeptide sequences SEQ ID NO:3 or 5, or (b) the complement of such nucleotide sequence. “Substantially complementary” polynucleotides can include at least one base pair mismatch, such that at least one nucleotide present on a second polynucleotide, however the two polynucleotides will still have the capacity to hybridize. For instance, the middle nucleotide of each of the two DNA molecules 5′-AGCAAATAT and 5′-ATATATGCT will not base pair, but these two polynucleotides are nonetheless substantially complementary as defined herein. Two polynucleotides are substantially complementary if they hybridize under hybridization conditions exemplified by 2×SSC (SSC: 150 mM NaCl, 15 mM trisodium citrate, pH 7.6) at 55° C. Substantially complementary polynucleotides for purposes of the present invention preferably share at least one region of at least 20 nucleotides in length which shared region has at least 60% nucleotide identity, preferably at least 80% nucleotide identity, more preferably at least 90% nucleotide identity and most preferably at least 95% nucleotide identity. Particularly preferred substantially complementary polynucleotides share a plurality of such regions.

Percent identity between two polypeptide or polynucleotide sequences is generally determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusove et al. (FEMS Microbiol. Lett., 174, 247-250 (1999)), and available on the world wide web at www.ncbi.nlm.nih.gov/gorf/b12.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.”

Likewise, two nucleotide sequences are preferably compared using the Blastn program, version 2.0.11, of the BLAST 2 search algorithm, also as described by Tatusova et al. (FEMS Microbiol. Lett, 174, 247-250 (1999)), and available on the world wide web at www.ncbi.nlm.nih.gov/blast.html. Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. Locations and levels of nucleotide sequence identity between two nucleotide sequences can also be readily determined using CLUSTALW multiple sequence alignment software (J. Thompson et al., Nucl. Acids Res., 22:4673-4680 (1994)), available at from the world wide web at www.ebi.ac.uk/clustalw/.

It should be understood that a polynucleotide that encodes a polypeptide represented by SEQ ID NOs: 3 or 5 is not limited to a polynucleotide that contains all or a portion of naturally occurring genomic or cDNA nucleotide sequence in S. clavuligerus, but also includes the class of polynucleotides that encode such polypeptides as a result of the degeneracy of the genetic code. For example, the naturally occurring nucleotide sequence SEQ ID NO:2 is but one member of the class of nucleotide sequences that encodes a polypeptide having amino acid SEQ ID NO:3; likewise with SEQ ID NO:4, that is but one member of the class of nucleotide sequences that encodes a polypeptide having amino acid SEQ ID NO:5. The class of nucleotide sequences that encode a selected polypeptide sequence is large but finite, and the nucleotide sequence of each member of the class can be readily determined by one skilled in the art by reference to the standard genetic code, wherein different nucleotide triplets (codons) are known to encode the same amino acid. Likewise, a polynucleotide of the invention that encodes a biologically active analog or subunit of the S. clavuligerus sensor kinase CbsA1 or the response regulator CbsA2 includes the multiple members of the class of polynucleotides that encode the selected polypeptide sequence.

Moreover, a polynucleotide that “encodes” a polypeptide of the invention optionally includes both coding and noncoding regions, and it should therefore be understood that, unless expressly stated to the contrary, a polynucleotide that “encodes” a polypeptide is not structurally limited to nucleotide sequences that encode a polypeptide but can include other nucleotide sequences outside (i.e., 5′ or 3′ to) the coding region.

The polynucleotides of the invention can be DNA, RNA, or a combination thereof, and can include any combination of naturally occurring, chemically modified or enzymatically modified nucleotides. As noted above, the polynucleotide can be equivalent to the polynucleotide encoding CbsA1 or CbsA2, or a constituent peptide, or it can include said polynucleotide in addition to one or more additional nucleotides. For example, the polynucleotide of the invention can be a vector, such as an expression or cloning vector. A vector useful in the present invention can be circular or linear, single-stranded or double-stranded, and can include DNA, RNA, or any modification or combination thereof. The vector can be a plasmid, a cosmid, or a viral vector, such as baculovirus. Preferably, the polynucleotide of the invention takes the form of an expression vector that is expressible in bacterial expression system such as E. coli, yeast, mammalian cell culture or insect cells, most preferably E. coli.

The invention further provides methods for making the CbsA1 and CbsA2 and constituent peptides of the invention (including analogs and biologically active subunits thereof), as well as methods for making the polynucleotides that encode them. The methods include biological, enzymatic, and chemical methods, as well as combinations thereof, and are well-known in the art. For example, CbsA1 or CbsA2 or constituent peptides can be expressed in a host cell from using standard recombinant DNA technologies; they can be enzymatically synthesized in vitro using a cell-free RNA based system; or they can be synthesized using chemical technologies such as solid phase peptide synthesis, as is well-known in the art. When recombinant DNA technologies are used, the host cell can be, for example, a bacterial cell, an insect cell, a yeast cell, or a mammalian cell.

In another aspect, the present invention provides a modified Streptomyces species, preferably a modified S. clavuligerus, in which the production or activity of either or both of a sensor kinase polypeptide encoded by cbsA1, or a response regulator polypeptide encoded by cbsA2, is reduced or eliminated. The modified Streptomyces of the invention, e.g., modified S. clavuligerus, preferably exhibits enhanced production of a bioactive metabolite such as clavulanic acid and/or cephamycin C when compared to levels of the bioactive metabolite produced by a comparable wild-type Streptomyces (e.g., wild-type S. clavuligerus). Thus, reduction or elimination of the activity of any one or more of these encoded polypeptides or RNAs is evidenced by an increase in the production of one or more bioactive metabolites in the Streptomyces species so modified, compared to wild-type levels of production of the bioactive metabolites in the Streptomyces species. The present invention also includes methods for making and using the modified Streptomyces species of the invention, preferably modified S. clavuligerus, as described in more detail below. The invention will be further described as it relates to S. clavuligerus, but it should be understood that other Streptomyces spp. are contemplated as well in all aspects of the invention as well.

Enhanced production of bioactive metabolites in S. clavuligerus can be achieved by reducing or eliminating, directly or indirectly, the production or activity of a polypeptide or RNA encoded by either or both of the genes involved in the two-component regulatory system described herein, cbsA1 or cbsA2. The present invention thus contemplates a method for increasing the production of one or more bioactive metabolites, such as those that are members of the cephalosporin family in S. clavuligerus. Methods for measuring the levels of bioactive metabolites produced by S. clavuligerus are well known to the art. For example, cephamycin C can be measured using the agar plate diffusion method with ESS E. coli strains as indicator microorganisms (Malmberg et al., 1993, J. Bacteriol. 175, 6916-24), and clavulanic acid can be measured using the β-lactamase inhibition assay with Klebsiella pneumomiae as indicator (Brown et al., 1976, J. Antibiot. (Tokyo) 29, 668-9).

In one embodiment, the modified S. clavuligerus capable of enhanced production of bioactive metabolites is a genetically modified S. clavuligerus, in which chromosomal DNA of the cbsA locus has been structurally altered, e.g., via random or site directed mutagenesis, as described in more detail below, so as to directly or indirectly reduce or eliminate the production of a gene product (i.e., an RNA or polypeptide) encoded by the cbsA1 gene or the cbsA2 gene. In another embodiment, the modified S. clavuligerus is an S. clavuligerus into which has been introduced an exogenous agent, such as a DNA, RNA or small molecule inhibitor, which directly or indirectly reduces or eliminates the production or activity of a gene product (i.e., an RNA or polypeptide) encoded by the cbsA1 gene or the cbsA2 gene,

Methods for reducing or eliminating the production or activity of one or more gene products of cbsA1 and/or cbsA2, thereby increasing the production of a selected bioactive metabolite, include, without limitation, those that act directly or indirectly on the cbsA1 and/or cbsA2 genes; those that act on directly or indirectly the mRNA transcript produced by one or both of those genes; those that affect the translation of the mRNA transcript(s) into a polypeptide; and those that inhibit the activity of the transcribed RNA or translated polypeptide.

For example, as alluded to above, one way to increase production of bioactive metabolites in S. clavuligerus is by structurally altering the cbsA1 gene (encoding a sensor kinase) and/or the cbsA2 gene (encoding a response regulator), or other region of the cbsA locus that affects expression of the cbsA1 and/or cbsA2 genes. Any portion of the gene can be altered, and structural alterations can be made in either or both of noncoding and coding portions of the gene. Structural alterations can be made, for example, the promoter regions, operator regions, other regulatory regions that are found upstream or downstream from the coding region, and/or in the coding region. Any method of mutation can be uses; for example, either or both of cbsA1 and cbsA2 can be mutated using random (e.g., ultraviolet radiation) or site-directed mutagenesis to introduce insertions, deletions and/or substitutions. Using insertional mutagenesis techniques well known to the art, for example, foreign DNA can be inserted into the cbsA1 gene and/or the cbsA2 gene to produce a genetically modified S. clavuligerus which is an insertional mutant characterized by enhanced production of a bioactive metabolite. Alternatively, transcription of a gene can be impeded by delivering to the cell an exogenous agent such as antisense DNA or RNA molecule or a double stranded RNA molecule, for example using RNA interference (RNAi); or by introducing into the cell DNAs operatively encoding such bioactive RNAs. In that embodiment, the S. clavuligerus so modified may not be genetically modified, but the introduction of the exogenous agent nonetheless acts to directly or indirectly reduce or eliminate the production or activity of one or more gene products of cbsA1 and/or cbsA2.

Another way the activity of a polypeptide can be reduced is by interfering with the mRNA transcription product of the gene. For example, an exogenous agent such as a ribozyme (or a DNA vector operably encoding a ribozyme) can be delivered to the cell to cleave the target mRNA, thereby reducing or eliminating production of the polypeptide. Antisense nucleic acids and double stranded RNAs may also be used to interfere with translation.

The polypeptide product of the cbsA1 and/or cbsA2 genes can also be targeted. Antibodies or antibody-like molecules such as peptide aptamers can be introduced into the cell to abolish the activity of the translated polypeptide, as can protein or peptide inhibitors such as linear or cyclic peptides, peptidomimetics, or small organic molecules. The sensor kinase activity of the polypeptide encoded by cbsA1, and/or the activity of the response regulator polypeptide encoded by cbsA2, can be reduced or eliminated to yield a modified S. clavuligerus which exhibits enhanced production of a bioactive metabolite. In general, the invention contemplates the use of any method that interferes with the production or activity of that cbsA 1 and/or cbsA2 gene, mRNA or encoded polypeptide so as to result in increased production of a bioactive metabolite to produce a modified S. clavuligerus.

In another aspect, the invention provides a method for producing a bioactive metabolite, such as a cephamycin or a clavulanic acid, by culturing a modified S. clavuligerus under conditions and for a time sufficient to allow production of a bioactive metabolite, followed by isolating the bioactive metabolite. The bioactive metabolite can be isolated at the end of the culture—period or during a continuous culture, as described in more detail below. As noted earlier, a modified S. clavuligerus that is capable of enhanced production of a bioactive metabolite can be a genetically modified S. clavuligerus, in which chromosomal DNA of the cbsA locus has been structurally altered, or it can be a S. clavuligerus into which has been introduced an exogenous agent, such as a DNA, RNA or small molecule inhibitor, which directly or indirectly reduces or eliminates the production or activity of a gene product (i.e., an RNA or polypeptide) encoded by the cbsA1 gene or the cbsA2 gene. In the latter case involving the use of an exogenous agent, the method of producing a bioactive metabolite optionally includes introducing the exogenous agent into the S. clavuligerus so as to directly or indirectly reduce or eliminate the production or activity of an RNA or polypeptide encoded by the cbsA1 gene or the cbsA2 gene, thereby causing increased production of the bioactive metabolite. In the former case involving the use of a genetically modified S. clavuligerus, the method optionally includes the step of genetically modifying the S. clavuligerus as described herein to yield a genetically modified S. clavuligerus capable of increased production of a bioactive metabolite.

Culture conditions for S. clavuligerus are well-known (see, e.g., Neto et al., Braz. J. Chem. Eng. vol. 22 no. 4 São Paulo October/December 2005). Culture methods that can be utilized include, but are not limited to, a batch culture, a continuous culture, or a “fed-batch” culture. A “continuous cell culture” or, simply, “continuous culture” is a cell culture characterized by both a continuous inflow of a liquid nutrient feed and a continuous liquid outflow. The nutrient feed may, but need not, be a concentrated nutrient feed. Continuously supplying a nutrient solution at about the same rate that cells are washed out of the reactor by spent medium allows maintenance of a culture in a condition of stable multiplication and growth. In a type of bioreactor known as a chemostat, the cell culture is continuously fed fresh nutrient medium, and spent medium, cells and excreted cell product are continuously drawn off. Alternatively, a continuous culture may constitute a “perfusion culture,” in which case the liquid outflow contains culture medium that is substantially free of cells, or substantially lower cell concentration than that in the bioreactor. In a perfusion culture, cells can be retained by, for example, filtration, centrifugation, or sedimentation. A fed-batch culture can be converted to a continuous culture by initiating a substantially continuous inflow of nutrients, which continuous inflow can take the form of a typical cell culture medium or a concentrated nutrient feed, concurrent with a substantially continuous outflow or other systematic removal of culture medium or cells.

A “fed-batch” culture is a batch cell culture to which substrate, in either solid or concentrated liquid form, is added either periodically or continuously during the run. Just as in a batch culture, a fed-batch culture is initiated by inoculating cells to the medium, but, in contrast to a batch culture, there is a subsequent inflow of nutrients, such as by way of a concentrated nutrient feed. In contrast to a continuous culture there is no systematic removal of culture fluid or cells from a fed-batch culture (i.e., there is no outflow in a fed-batch culture). Use of a fed-batch culture is advantageous in applications that involve monitoring and manipulating the levels of various analytes in the culture medium, since the concentrations of nutrients and metabolites in culture medium can be readily controlled or affected by altering the composition of the nutrient feed. The nutrient feed delivered to a fed-batch culture is typically a concentrated nutrient solution containing an energy source, e.g., carbohydrates; optionally, the concentrated nutrient solution delivered to a fed-batch culture can contain amino acids, lipid precursors and/or salts. In a fed-batch culture, this nutrient feed is typically rather concentrated to minimize the increase in culture volume while supplying sufficient nutrients for continued cell growth.

Cultured cells are preferably in a “suspension state.” A “suspension” of cells is to be broadly understood as including all types of suspended or dispersed cell cultures; the term “suspension state” is thus used to distinguish cells that are not cultured in a liquid medium, such as cells cultured by way of adhering on a Petri dish. Thus, the term “suspension” includes both freely dispersed cells and agglomerated cells, regardless of whether agglomeration occurs spontaneously or as a result of some exogenously supplied nucleating factor or agent. Cultured cells according to the invention also, however, are not limited to cells in suspension but can also include adherent cultures such as plate cultures. Optionally the cultured cells can be harvested during or subsequent to the period of continuous culture.

The bioactive metabolite, such as an antibiotic (e.g. cephamycin C) or a β-lactamase inihibitor (e.g., clavulanic acid) that is produced in the cell culture is isolated from culture broth (or supernatant) using protocols, methods and techniques that are well-known in the art. For example, clavulanic acid can be isolated by filtration and chromatographic purification as illustrated in GB 1,508,977 prior to the extraction of the aqueous solution with the organic solvent to obtain a solution of impure clavulanic acid in an organic solvent or by continuous adsorption process (Almeida et al., 2003 Appl. Biochem. Biotechnol., 105-108:867-79). The method of the invention is to be broadly understood to include the production and isolation of any or all bioactive metabolites recovered or recoverable from the modified S. clavuligerus cells of the invention, regardless of whether the bioactive metabolites are members of the cephalosporin family.

The detailed kinetic analysis set forth in the following example shows that the effect on antibiotic production when the cbsA locus is altered through gene disruption. This general strategy can now be applied to the more than 50 two-component regulatory systems that are likely present in the S. clavuligerus genome.

The present invention is illustrated by the following example. It is to be understood that the particular examples, materials, amounts, and procedures are to be interpreted broadly in accordance with the scope and spirit of the invention as set forth herein.

### EXAMPLE

### A Two-Component Regulator from Streptomyces clavuligerus affects Both Cephamycin C and Clavulanic Acid Production

We report the identification of a two-component regulatory gene locus (cbsA) from S. clavuligerus. Disruption mutants of the response regulator and sensor kinase were constructed and respectively revealed positive affects on both cephamycin C and clavulanic acid production in liquid cultures.

Constructing a cosmid library and screening for absA2 homologous genes. S. clavuligerus NRRL 3585 (ATCC 27064) was used in this study. A S. clavuligerus genome library was constructed using the pNJ1 cosmid vector (Tuan et al., 1990, Gene 90, 21-9) and with E. coli DH5a as the host strain. About 2000 recombinant colonies were screened with a probe obtained by PCR amplification of an S. clavuligerus absA-homologous gene to obtain 18 positive clones. After restriction enzyme digestion of DNA isolated from the positive clones, followed by Southern hybridization with the same probe, five cosmids were confirmed to contain an absA2-homologous gene fragment. The gene fragments identified by Southern hybridization were then subcloned into the pBlueScript KS(−) vector for subsequent sequencing. The complete sequence for each ORF was obtained using a primer-walking strategy.

Analysis of the cbsA locus revealed that the two-component system is composed of a response regulator gene—cbsA2, and a sensor kinase gene —cbsA1, downstream of cbsA2 (FIG. 1A). The order in which cbsA2 and cbsA1 resides on the transcript is reversed of the order in the absA1/2 two-component system in S. coelicolor (Champness, 1992, Gene 115, 55-60). CbsA2 contains two conserved domains as most other response regulators do—a receiver domain and an output domain (DNA binding domain in this case). On the other hand, CbsA1 contains transmembrane domains, H box, N box and G box, which are characteristic of histidine sensor kinase of two-component systems. Five transmembrane helices was revealed by TMPRED program (www.ch.embnet.org/software/TMPRED_form.html), indicating that like many other sensor kinase, CbsA1 resides in the membrane and is responsible for sensing environmental signals.

Insertional disruption of cbsA2 and cbsA1. Plasmids for disruption of cbsA2 and cbsA1 genes were constructed and transformed into S. clavuligerus NRRL 3585 protoplasts (FIG. 2). Double crossover homologous recombination was carried out for disruption of cbsA1 and cbsA2 genes, respectively. Disruption plasmids were constructed using pHJL400 (Larson et al., 1986, Plasmid 15, 199-209) with a kanamycin resistance gene flanked on each side by two 700 bp fragments bearing sequences identical to the targeted genes.

Transformed protoplasts were spread onto R2YEG plates and after 42 hours the plates were overlaid with P buffer (Malmberg et al., 1993, J. Bacteriol. 175, 6916-24) containing thiostrepton and kanamycin at a final concentration of 5 μg/ml and 50 μg/ml,. respectively. Transformants were allowed to sporulate on TOA agar medium containing only kanamycin. Spores from the first generation of transformants were plated out on kanamycin containing agar and replicated onto agar containing both kanamycin and thiostrepton. Approximately 30% of the colonies appeared to grow only on kanamycin but not thiostrepton containing agar, and were thus considered putative mutants. To obtain mutants derived from double crossover homologous recombination, we proceeded for two additional generations of culture propagation and antibiotic selection.

Disruptions were confirmed by both PCR amplification of the region spanning the mutation using chromosomal DNA from the mutant strain as template, and by Southern blot analysis. Two mutant strains were identified corresponding to DHS3507 for ΔcbsA2::kan and DHS3508 for ΔcbsA1::kan.

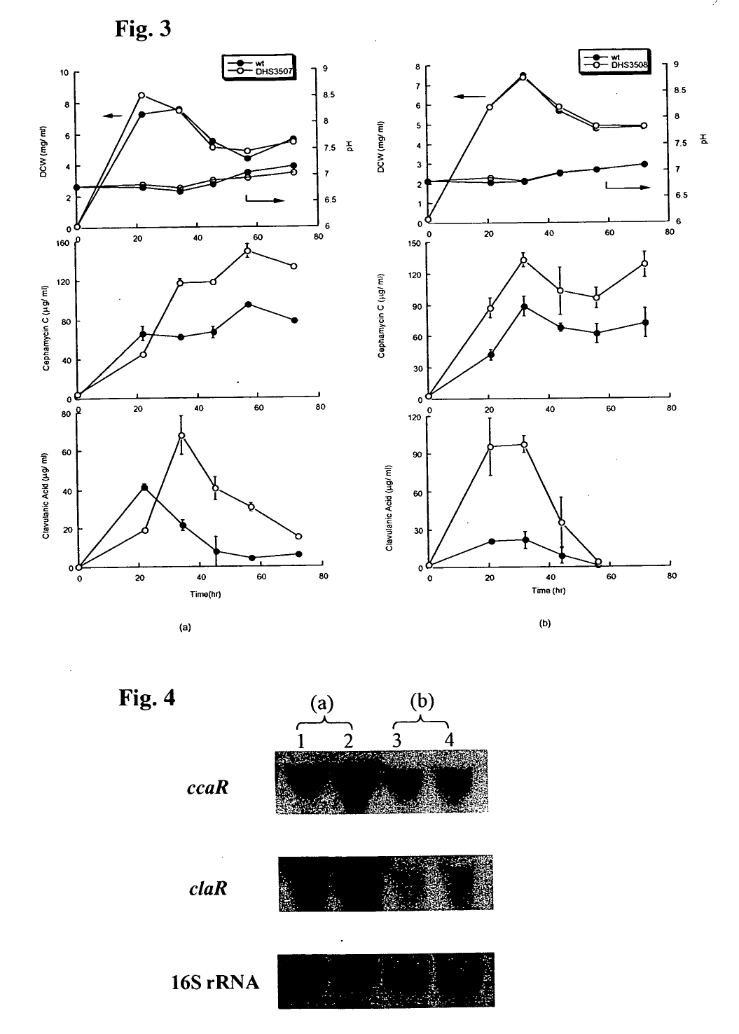

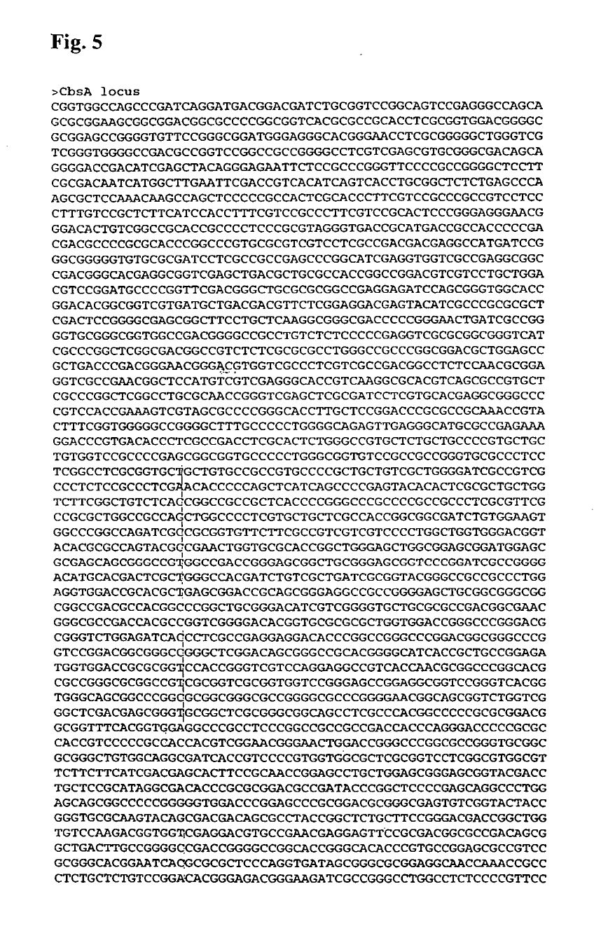

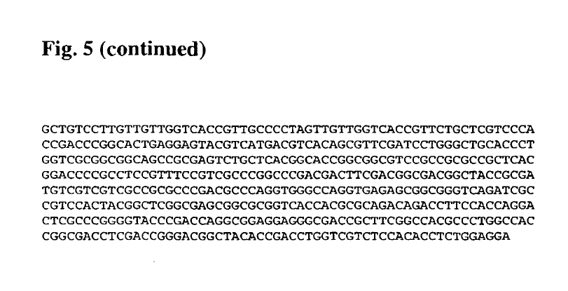

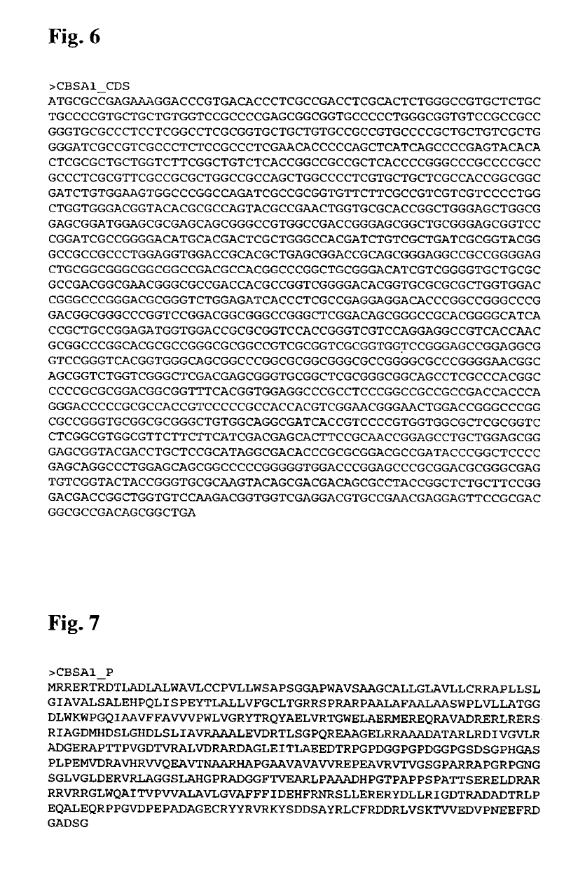

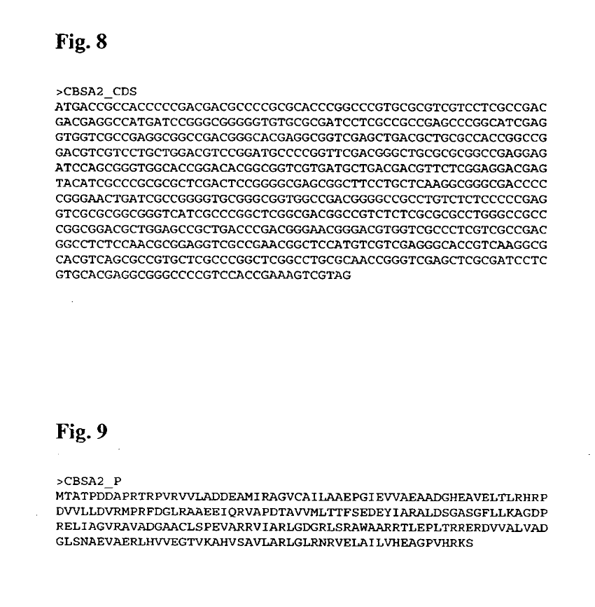

Increased production of cephamycin C and clavulanic acid. To compare antibiotic production, experiments were conducted separately for the two mutants to compare with wild type S. clavuligerus (FIG. 3(a, b)). Spores were produced on tomato oatmeal agar (TOA) (20 g tomato paste, 20 g oatmeal, 25 g Bacto Agar, 1 liter water, adjusted to pH 6.8) at 26° C. Stocks of spores were stored in 50% glycerol at −20° C. Liquid cultures were carried out at 28° C. in 250 ml baffled flasks containing 50 ml modified TSB medium (per liter medium containing 15 g TSB, 10 g glycerol, and 20 g MOPS). Seed cultures were carried out for 42 hours after inoculation of spores. Then 4% (v/v) of the seed culture was inoculated into the antibiotic production culture. Thiostrepton (5 μg/ml) and kanamycin (50 μg/ml) were added to the culture media if the strain carried the corresponding antibiotic resistance marker. R2YEG medium was used to regenerate S. clavuligerus protoplasts. In each experiment, 15 flasks were dedicated to each strain and among them 3 flasks were analyzed for each time point to obtain statistically significant data. Cephamycin was analyzed by the agar plate diffusion method with ESS E. coli strains as indicator microorganisms (Malmberg et al., 1993, J. Bacteriol. 175, 6916-24). Clavulanic acid was determined by the β-lactamase inhibition assay with Klebsiella pneumoniae as indicator (Brown et al., 1976, J. Antibiot. (Tokyo) 29, 668-9). For each sample, bioassay was conducted three times and the average value was taken as the measurement value. Alternatively, the imidazole reagent method was also used to detect clavulanic acid (Bird et al., 1982, Analyst 107, 1241-1245) and its reaction product had a maximum absorbance at 320 nm that was detected by UV spectrometry.

The data shows that the growth characteristics remained same for the wild-type strain and the mutant strains. Cephamycin C was more stable during the culture than clavulanic acid whose concentration dropped drastically after 40 hours. The peak values for both cephamycin C and clavulanic acid production of the mutants were enhanced nearly two-fold compared to wild-type S. clavuligerus. This finding is consistent with the observation of the absA replacement S. coelicolor mutant strain in plate-grown cultures, whose production of actinorhodin increased 5-fold while undecylprodigiosin increased 8-fold compared to the wild type strain (Brian et al., 1996, J. Bacteriol. 178, 3221-31).

Increased transcriptional expression of ccaR and claR genes. RNA was isolated from S. clavuligerus wild type and the two cbsA mutant strains from liquid culture in the modified TSB medium. Mycelia were quickly pelleted and the supernatant was aspirated completely. The samples were subjected to RNA isolation immediately or stored at −80° C. Frozen cell pellets were ground into a fine powder in the presence of liquid nitrogen and lysed with GIT buffer. The lysate was centrifuged and then layered onto a CsCl gradient cushion. The following steps were continued as described in Sambrook et al. (1989, Molecular Cloning. A Laboratory Manual, Cold Spring Harbor Laboratory Press, Cold Spring Harbor, N.Y.).

Extracted RNA was loaded into a denaturing formaldehyde agarose gel. After electrophoresis, RNA were blotted onto nylon filters and hybridized overnight at 42° C. with [³²P]dCTP-labelled probes internal to ccaR and claR in the buffer containing 50% formamide, 5×SSPE, 2× Denhardts solution (Sambrook et al., 1989, Molecular Cloning. A Laboratory Manual, Cold Spring Harbor Laboratory Press, Cold Spring Harbor, N.Y.) and 1% SDS. The 772 bp probe for ccaR mRNA was PCR amplified with 5′-GTGACGATCCGGCTCCTGGG-3′ (SEQ ID NO:6) and 5′-TTCAGGCCGGGGTACCGACC-3′ (SEQ ID NO:7) primers. The 5′-CGAGCAGACTCGTGGTGCGG-3′ (SEQ ID NO:8) and 5′-TCATGGAAGTGGCCCGGCGG-3′ (SEQ ID NO:9) primers were used to obtain a 761 bp long fragment for probing claR mRNA. The filters were washed with 1×SSC containing 0.1% SDS (sodium dodecyl sulfate) at room temperature (RT) for 15 minutes and then washed again with 0.2×SSC containing 0.1% SDS at 65° C. for 30 minutes.

RNA was isolated from cultures of the DHS3507 and DHS3508 cbsA mutant strains described above. Northern blotting with ccaR and claR probes indicated that the transcripts of both pathway-specific regulators were enhanced in the strains containing disruption of the cbsA locus (FIG. 4). This data concurs with the enhanced production of the both cephamycin C and clavulanic acid metabolites. CcaR has been shown to play a key role for control of claR (Perez-Redondo et al., 1998, Gene 211, 311-21). Introduction of additional copies of ccaR into S. clavuligerus promoted not only cephamycin C production but also clavulanic acid production (Perez-Llarena et al., 1997, J. Bacteriol.179, 2053-9). Therefore, in the cbsA mutants strains the increased level of claR transcripts might be caused by higher levels of CcaR. Of course, it does not exclude the possibility that factors other than CcaR are affecting claR expression.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### ADDITIONAL REFERENCE

Alexander et al. (1998) J. Bacteriol. 180, 4068-79
Anderson et al. (1999) Microbiol. 145, 2343-53
Bentley et al. (2002) Nature 417, 141-7
Bibb (1996) Microbiol. 142 (Pt 6), 1335-44
Bignell et al. (2005) Antimicrob. Agents Chemother. 49:1529-1541
Bird et al. (1982) Analyst 107, 1241-1245
Brian et al. (1996). J. Bacteriol. 178, 3221-31
Brown et al. (1976) J Antibiot. (Tokyo) 29, 668-9
Champness (1992) Gene 115, 55-60
Champness et al., U.S. Pat. No. 5,876,987, issued Mar. 2, 1999
Chang et al. (1996) Mol. Microbiol. 21, 1075-85
Chater (2001) Curr. Opin. Microbiol. 4, 667-73
Cundliffe et al. (2001). Antonie Van Leeuwenhoek 79, 229-34
Floriano et al. (1996) Mol. Microbiol. 21, 385-96
Hodgson (2000) Adv. Microb. Physiol. 42, 47-238
Hong et al. (2004) Mol. Microbiol. 52, 1107-21
Ishizuka et al. (1992) J. Bacteriol. 174, 7585-94
Jin et al. (2004) Microbiol. 150:1485-1493
Kormanec et al. (2000) Folia Microbiol. 45, 397-406
Larson et al. (1986) Plasmid 15, 199-209
Malmberg et al. (1993) J. Bacteriol. 175, 6916-24
Paget et al. (1999) Mol. Microbiol. 33, 97-107
Perez-Llarena et al. (1997). J. Bacteriol.179, 2053-9
Perez-Redondo et al. (1998) Gene 211, 311-21
Rolinson (1982) S. Af. Med. J. 62, 3A-4A
Ryding et al. (2002). J. Bacteriol. 184, 794-805
Sambrook et al. (1989) Molecular Cloning. A Laboratory Manual, Cold Spring Harbor Laboratory Press, Cold Spring Harbor, N.Y.
Sola-Landa et al. (2003) Proc. Natl. Acad. Sci. U S A 100, 6133-8
Tsujibo al. (1999) FEMS Microbiol. Lett. 181, 83-90
Tuan et al. (1990) Gene 90, 21-9
Ward, J. M. and Hodgson, J. E. (1993) FEMS Microbiol. Lett. 110, 239-42

What is claimed is:

1. A genetically modified Streptomyces comprising a structural alteration in the cbsA locus that reduces or eliminates the production or activity of a gene product of either or both of cbsA1 or cbsA2, which genetically modified Streptomyces exhibits enhanced production of a bioactive metabolite compared to the level of production of the bioactive metabolite in a wild-type Streptomyces.

2. The genetically modified Streptomyces of claim 1 wherein the structural alteration comprises a deletion, insertion or substitution in the coding region of either or both of cbsA1 or the cbsA2.

3. The genetically modified Streptomyces of claim 1 wherein the bioactive metabolite is selected from the group consisting of a cephamycin and a clavulanic acid.

4. The genetically modified Streptomyces of claim 1 wherein the Streptomyces is an S. clavuligerus.

5. A method for making a bioactive metabolite comprising:
culturing the genetically modified Streptomyces of claim 1 under conditions and for a time sufficient to produce the bioactive metabolite; and
isolating the bioactive metabolite.

6. The method of claim 5 wherein the bioactive metabolite is selected from the group consisting of a cephamycin and a clavulanic acid.

7. The method of claim 5 wherein the Streptomyces is an S. clavuligerus.

8. A method for making a bioactive metabolite comprising:
introducing an exogenous agent into a Streptomyces to yield a modified Streptomyces, wherein the exogenous agent reduces or eliminates the production or activity of a gene product of either or both of cbsA1 or cbsA2;
culturing the modified Streptomyces under conditions and for a time sufficient to produce the bioactive metabolite; and
isolating the bioactive metabolite.

9. The method of claim 8 wherein the bioactive metabolite is selected from the group consisting of a cephamycin and a clavulanic acid.

10. The method of claim 8 wherein the Streptomyces is an S. clavuligerus.

11. A method for making a modified Streptomyces comprising modifying a Streptomyces cell to reduce or eliminate the production or activity of a gene product of either or both of cbsA1 or cbsA2.

12. The method of claim 11 wherein the modified Streptomyces cell exhibits enhanced production of a bioactive metabolite compared to the level of production of the bioactive metabolite in a wild-type Streptomyces cell.

13. The method of claim 11 wherein the bioactive metabolite is selected from the group consisting of a cephamycin and a clavulanic acid.

14. The method of claim 11 wherein the Streptomyces is S. clavuligerus.

15. The method of claim 11 where modifying the Streptomyces cell comprises structurally altering the cbsA locus to yield a genetically modified Streptomyces.

16. The method of claim 11 wherein modifying the Streptomyces cell comprises introducing an exogenous agent into the cell, wherein the exogenous agent reduces or eliminates the production or activity of a gene product of cbsA1 gene or the cbsA2 gene.

17. A genetically modified S. clavuligerus comprising a structural alteration in the cbsA locus that reduces or eliminates the production or activity of a gene product of either or both of cbsA1 or cbsA2, which genetically modified S. clavuligerus exhibits enhanced production of a cephamycin or a clavulanic acid compared to the level of production of the cephamycin or clavulanic acid in a wild-type S. clavuligerus.

18. A method for producing a bioactive metabolite in S. clavuligerus comprising reducing or eliminating the production or activity of an RNA or polypeptide gene product encoded by cbsA1 or cbsA2, wherein said reduction or elimination in the production of activity of the gene product results in increased production of the bioactive metabolite.

19. An isolated polypeptide comprising an amino acid sequence having at least 80% identity to SEQ ID NO: 3 or SEQ ID NO:5.

20. The isolated polypeptide of claim 19 comprising an amino acid sequence having at least 90% identity to SEQ ID NO: 3 or SEQ ID NO:5

21. The isolated polypeptide of claim 19 comprising SEQ ID NO:3 or SEQ ID NO:5.

22. An isolated polynucleotide that encodes a polypeptide comprising an amino acid sequence having at least 80% identity to SEQ ID NO: 3 or SEQ ID NO:5.

23. An isolated polynucleotide comprising a nucleotide sequence encoding SEQ ID NO:3 or SEQ ID NO:5, or its complement.

24. The isolated polynucleotide of claim 23 comprising SEQ ID NO: 2 or SEQ ID NO:4, or the complement of SEQ ID NO: 2 or SEQ ID NO:4.

25. An isolated polynucleotide comprising a nucleotide sequence having at least 90% identity to SEQ ID NO:2 or SEQ ID NO:4.


SEQUENCE LISTING (9 sequences)

SEQ ID NO: 1 - DNA, 3597 bp, streptomyces clavuligerus
cggtggccagcccgatcaggatgacggacgatctgcggtccggcagtccgagggccagcagcgcggaagcggcggacggcgccccggcggtcacgcgccgcacctcgcggtggacggggcgcggagccggggtgttccgggcggatgggagggcacgggaacctcgcgggggctgggtcgtcgggtggggccgacgccggtccggccgccggggcctcgtcgagcgtgcgggcgacagcaggggaccgacatcgagctacagggagaattctccgcccgggttccccgccggggctccttcgcgacaatcatggcttgaattcgaccgtcacatcagtcacctgcggctctctgagcccaagcgctccaaacaagccagctcccccgccactcgcacccttcgtccgcccgccgtcctccctttgtccgctcttcatccacctttcgtccgcccttcgtccgcactcccgggagggaacgggacactgtcggccgcaccgcccctcccgcgtagggtgaccgcatgaccgccacccccgacgacgccccgcgcacccggcccgtgcgcgtcgtcctcgccgacgacgaggccatgatccgggcgggggtgtgcgcgatcctcgccgccgagcccggcatcgaggtggtcgccgaggcggccgacgggcacgaggcggtcgagctgacgctgcgccaccggccggacgtcgtcctgctggacgtccggatgccccggttcgacgggctgcgcgcggccgaggagatccagcgggtggcaccggacacggcggtcgtgatgctgacgacgttctcggaggacgagtacatcgcccgcgcgctcgactccggggcgagcggcttcctgctcaaggcgggcgacccccgggaactgatcgccggggtgcgggcggtggccgacggggccgcctgtctctcccccgaggtcgcgcggcgggtcatcgcccggctcggcgacggccgtctctcgcgcgcctgggccgcccggcggacgctggagccgctgacccgacgggaacgggacgtggtcgccctcgtcgccgacggcctctccaacgcggaggtcgccgaacggctccatgtcgtcgagggcaccgtcaaggcgcacgtcagcgccgtgctcgcccggctcggcctgcgcaaccgggtcgagctcgcgatcctcgtgcacgaggcgggccccgtccaccgaaagtcgtagcgccccgggcaccttgctccggacccgcgccgcaaaccgtactttcggtgggggccggggctttgccccctggggcagagttgagggcatgcgccgagaaaggacccgtgacaccctcgccgacctcgcactctgggccgtgctctgctgccccgtgctgctgtggtccgccccgagcggcggtgccccctgggcggtgtccgccgccgggtgcgccctcctcggcctcgcggtgctgctgtgccgccgtgccccgctgctgtcgctggggatcgccgtcgccctctccgccctcgaacacccccagctcatcagccccgagtacacactcgcgctgctggtcttcggctgtctcaccggccgccgctcaccccgggcccgccccgccgccctcgcgttcgccgcgctggccgccagctggcccctcgtgctgctcgccaccggcggcgatctgtggaagtggcccggccagatcgccgcggtgttcttcgccgtcgtcgtcccctggctggtgggacggtacacgcgccagtacgccgaactggtgcgcaccggctgggagctggcggagcggatggagcgcgagcagcgggccgtggccgaccgggagcggctgcgggagcggtcccggatcgccggggacatgcacgactcgctgggccacgatctgtcgctgatcgcggtacgggccgccgccctggaggtggaccgcacgctgagcggaccgcagcgggaggccgccggggagctgcggcgggcggcggccgacgccacggcccggctgcgggacatcgtcggggtgctgcgcgccgacggcgaacgggcgccgaccacgccggtcggggacacggtgcgcgcgctggtggaccgggcccgggacgcgggtctggagatcaccctcgccgaggaggacacccggccgggcccggacggcgggcccggtccggacggcgggccgggctcggacagcgggccgcacggggcatcaccgctgccggagatggtggaccgcgcggtccaccgggtcgtccaggaggccgtcaccaacgcggcccggcacgcgccgggcgcggccgtcgcggtcgcggtggtccgggagccggaggcggtccgggtcacggtgggcagcggcccggcgcggcgggcgccggggcgcccggggaacggcagcggtctggtcgggctcgacgagcgggtgcggctcgcgggcggcagcctcgcccacggcccccgcgcggacggcggtttcacggtggaggcccgcctcccggccgccgccgaccacccagggacccccgcgccaccgtcccccgccaccacgtcggaacgggaactggaccgggcccggcgccgggtgcggcgcgggctgtggcaggcgatcaccgtccccgtggtggcgctcgcggtcctcggcgtggcgttcttcttcatcgacgagcacttccgcaaccggagcctgctggagcgggagcggtacgacctgctccgcataggcgacacccgcgcggacgccgatacccggctccccgagcaggccctggagcagcggcccccgggggtggacccggagcccgcggacgcgggcgagtgtcggtactaccgggtgcgcaagtacagcgacgacagcgcctaccggctctgcttccgggacgaccggctggtgtccaagacggtggtcgaggacgtgccgaacgaggagttccgcgacggcgccgacagcggctgacttgccggggccgaccggggccggcaccgggcacacccgtgccggagcgccgtccgcgggcacggaatcacgcgcgctcccaggtgatagcgggcgcggaggcaaccaaaccgccctctgctctgtccggacacgggagacgggaagatcgccgggcctggcctctccccgttccgctgtccttgttgttggtcaccgttgcccctagttgttggtcaccgttctgctcgtcccaccgacccggcactgaggagtacgtcatgacgtcacagcgttcgatcctgggctgcaccctggtcgcggcggcagccgcgagtctgctcacggcaccggcggcgtccgccgcgccgctcacggaccccgcctccgtttccgtcgcccggcccgacgacttcgacggcgacggctaccgcgatgtcgtcgtcgccgcgcccgacgcccaggtgggccaggtgagagcggcgggtcagatcgccgtccactacggctcggcgagcggcgcggtcaccacgcgcagacagaccttccaccaggactcgcccggggtacccgaccaggcggaggagggcgaccgcttcggccacgccctggccaccggcgacctcgaccgggacggctacaccgacctggtcgtctccacacctctggagga

SEQ ID NO: 2 - DNA, 1638 bp, streptomyces clavuligerus
atgcgccgagaaaggacccgtgacaccctcgccgacctcgcactctgggccgtgctctgctgccccgtgctgctgtggtccgccccgagcggcggtgccccctgggcggtgtccgccgccgggtgcgccctcctcggcctcgcggtgctgctgtgccgccgtgccccgctgctgtcgctggggatcgccgtcgccctctccgccctcgaacacccccagctcatcagccccgagtacacactcgcgctgctggtcttcggctgtctcaccggccgccgctcaccccgggcccgccccgccgccctcgcgttcgccgcgctggccgccagctggcccctcgtgctgctcgccaccggcggcgatctgtggaagtggcccggccagatcgccgcggtgttcttcgccgtcgtcgtcccctggctggtgggacggtacacgcgccagtacgccgaactggtgcgcaccggctgggagctggcggagcggatggagcgcgagcagcgggccgtggccgaccgggagcggctgcgggagcggtcccggatcgccggggacatgcacgactcgctgggccacgatctgtcgctgatcgcggtacgggccgccgccctggaggtggaccgcacgctgagcggaccgcagcgggaggccgccggggagctgcggcgggcggcggccgacgccacggcccggctgcgggacatcgtcggggtgctgcgcgccgacggcgaacgggcgccgaccacgccggtcggggacacggtgcgcgcgctggtggaccgggcccgggacgcgggtctggagatcaccctcgccgaggaggacacccggccgggcccggacggcgggcccggtccggacggcgggccgggctcggacagcgggccgcacggggcatcaccgctgccggagatggtggaccgcgcggtccaccgggtcgtccaggaggccgtcaccaacgcggcccggcacgcgccgggcgcggccgtcgcggtcgcggtggtccgggagccggaggcggtccgggtcacggtgggcagcggcccggcgcggcgggcgccggggcgcccggggaacggcagcggtctggtcgggctcgacgagcgggtgcggctcgcgggcggcagcctcgcccacggcccccgcgcggacggcggtttcacggtggaggcccgcctcccggccgccgccgaccacccagggacccccgcgccaccgtcccccgccaccacgtcggaacgggaactggaccgggcccggcgccgggtgcggcgcgggctgtggcaggcgatcaccgtccccgtggtggcgctcgcggtcctcggcgtggcgttcttcttcatcgacgagcacttccgcaaccggagcctgctggagcgggagcggtacgacctgctccgcataggcgacacccgcgcggacgccgatacccggctccccgagcaggccctggagcagcggcccccgggggtggacccggagcccgcggacgcgggcgagtgtcggtactaccgggtgcgcaagtacagcgacgacagcgcctaccggctctgcttccgggacgaccggctggtgtccaagacggtggtcgaggacgtgccgaacgaggagttccgcgacggcgccgacagcggctga

SEQ ID NO: 3 - protein, 545 aa, streptomyces clavuligerus
MRRERTRDTLADLALWAVLCCPVLLWSAPSGGAPWAVSAAGCALLGLAVLLCRRAPLLSLGIAVALSALEHPQLISPEYTLALLVFGCLTGRRSPRARPAALAFAALAASWPLVLLATGGDLWKWPGQIAAVFFAVVVPWLVGRYTRQYAELVRTGWELAERMEREQRAVADRERLRERSRIAGDMHDSLGHDLSLIAVRAAALEVDRTLSGPQREAAGELRRAAADATARLRDIVGVLRADGERAPTTPVGDTVRALVDRARDAGLEITLAEEDTRPGPDGGPGPDGGPGSDSGPHGASPLPEMVDRAVHRVVQEAVTNAARHAPGAAVAVAVVREPEAVRVTVGSGPARRAPGRPGNGSGLVGLDERVRLAGGSLAHGPRADGGFTVEARLPAAADHPGTPAPPSPATTSERELDRARRRVRRGLWQAITVPVVALAVLGVAFFFIDEHFRNRSLLERERYDLLRIGDTRADADTRLPEQALEQRPPGVDPEPADAGECRYYRVRKYSDDSAYRLCFRDDRLVSKTVVEDVPNEEFRDGADSG

SEQ ID NO: 4 - DNA, 696 bp, streptomyces clavuligerus
atgaccgccacccccgacgacgccccgcgcacccggcccgtgcgcgtcgtcctcgccgacgacgaggccatgatccgggcgggggtgtgcgcgatcctcgccgccgagcccggcatcgaggtggtcgccgaggcggccgacgggcacgaggcggtcgagctgacgctgcgccaccggccggacgtcgtcctgctggacgtccggatgccccggttcgacgggctgcgcgcggccgaggagatccagcgggtggcaccggacacggcggtcgtgatgctgacgacgttctcggaggacgagtacatcgcccgcgcgctcgactccggggcgagcggcttcctgctcaaggcgggcgacccccgggaactgatcgccggggtgcgggcggtggccgacggggccgcctgtctctcccccgaggtcgcgcggcgggtcatcgcccggctcggcgacggccgtctctcgcgcgcctgggccgcccggcggacgctggagccgctgacccgacgggaacgggacgtggtcgccctcgtcgccgacggcctctccaacgcggaggtcgccgaacggctccatgtcgtcgagggcaccgtcaaggcgcacgtcagcgccgtgctcgcccggctcggcctgcgcaaccgggtcgagctcgcgatcctcgtgcacgaggcgggccccgtccaccgaaagtcgtag

SEQ ID NO: 5 - protein, 231 aa, streptomyces clavuligerus
MTATPDDAPRTRPVRVVLADDEAMIRAGVCAILAAEPGIEVVAEAADGHEAVELTLRHRPDVVLLDVRMPRFDGLRAAEEIQRVAPDTAVVMLTTFSEDEYIARALDSGASGFLLKAGDPRELIAGVRAVADGAACLSPEVARRVIARLGDGRLSRAWAARRTLEPLTRRERDVVALVADGLSNAEVAERLHVVEGTVKAHVSAVLARLGLRNRVELAILVHEAGPVHRKS

SEQ ID NO: 6 - DNA, 20 bp, artificial
  Primer
gtgacgatccggctcctggg

SEQ ID NO: 7 - DNA, 20 bp, artificial
  Primer
ttcaggccggggtaccgacc

SEQ ID NO: 8 - DNA, 20 bp, artificial
  Primer
cgagcagactcgtggtgcgg

SEQ ID NO: 9 - DNA, 20 bp, artificial
  Primer
tcatggaagtggcccggcgg